(19) United States
(12) Patent Application Publication
(10) Pub. No.: US 2011/0021738 A1
(43) Pub. Date: Jan. 27, 2011

(54) METHOD FOR THE PRODUCTION OF POLYOLS

(75) Inventor: Klaus Lorenz, Dormagen (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/919,956

(22) PCT Filed: Feb. 17, 2009
(86) PCT No.: PCT/EP09/01084

§ 371 Date: Aug. 27, 2010

(30) Foreign Application Priority Data
Feb. 28, 2008 (DE) 102008011683.1

(51) Int. Cl. C08G 18/32; C07C 43/10

(52) U.S. Cl. 528/77; 568/619

(57) ABSTRACT

The present invention relates to polyols obtainable via a simple process. Unless specified explicitly, polyols in the following are to be understood as meaning both polyether polyols and polyether ester polyols. The invention also provides the simple process itself and the use of the polyols according to the invention for the preparation of polyurethane materials.

The present invention provides polyols which contain soluble salts of monobasic inorganic acids and are obtainable via a process with no working up step. Unless specified explicitly, polyols in the following are to be understood as meaning both polyether polyols and polyether ester polyols. The invention also provides the process itself with no working up step and the use of the polyols according to the invention for the preparation of polyurethane materials.

Polymers which are suitable for the preparation of polyurethane materials, such as flexible or rigid foams or solid materials, such as elastomers, are in general obtained by polymerization of suitable alkylene oxides on polyfunctional starter compounds, i.e. those containing several zerewitinoff-active hydrogen atoms. For carrying out these polymerization reactions, the most diverse processes have been known for a long time, some of which complement each other:

On the one hand the base-catalysed addition of alkylene oxides on to starter compounds having zerewitinoff-active hydrogen atoms is of great industrial importance, and on the other hand the use of double metal cyanide compounds (“DMC catalysts”) is increasingly gaining importance for carrying out this reaction. With the use of highly active DMC catalysts, which are described e.g. in U.S. Pat. No. 5,470,813, EP-A 700 949, EP-A 743 093, EP-A 761 708, WO 97/40086, WO 98/16310 and WO 00/47649, preparation of polyether polyols at very low catalyst concentrations (25 ppm or less) is possible, so that it is no longer necessary to separate off the catalyst from the finished product. However, these catalysts are not suitable for the preparation of short-chain polyols or of polyols with high ethylene oxide contents. Polyethers based on starters containing amino groups also cannot be obtained via DMC catalysis. The basic catalysts known for a long time, e.g. based on alkali metal hydroxides, in contrast allow problem-free preparation of polyols which have short-chains and/or have a high ethylene oxide content and also of those containing amino groups, but as a general rule the catalyst must be removed from the alkaline crude polymer by means of a separate working up step. The addition of alkylene oxides on to suitable starter compounds catalysed by (Lewis) acids is of minor importance because of the by-product formation.

The base-catalysed addition of alkylene oxides, such as, for example, ethylene oxide or propylene oxide, on to starter compounds having zerewitinoff-active hydrogen atoms is carried out, as already mentioned, in the presence of alkali metal hydroxides, but alkali metal hydrides, alkali metal carboxylates, alkaline earth metal hydroxides or amines, such as N,N-dimethylbenzylamine or imidazole or imidazole derivatives, can also be employed. After the addition of the alkylene oxides has taken place, the polymerization-active centres on the polyether chains must be deactivated. Various procedures are possible for this. For example, neutralization can be carried out with dilute mineral acids, such as sulfuric acid or phosphoric acid. The strength of the second dissociation stage of sulfuric acid is sufficient to protonate the alkali metal hydroxides formed by hydrolysis of the active alcoholate groups, so that 2 mol of alcoholate groups can be neutralized per mol of sulfuric acid employed. Phosphoric acid, in contrast, must be employed in an amount equimolar to the amount of alcoholate groups to be neutralized. The salts formed during the neutralization and/or during the distilling off of the water must be separated off by means of filtration processes. Distillation and filtration processes are time- and energy-intensive and furthermore in some cases, e.g. in the case of polyols having C-2 units (ethylene oxide contents) in the chain, are not readily reproducible. Many processes which manage without a filtration step and in many cases also without a distillation step have therefore been developed: Neutralization with hydroxycarboxylic acids, such as lactic acid, is described in WO 98/20061 and US-A 2004167316 for the working up of short-chain polyols for rigid foam uses, and this is a widespread and well-established process. U.S. Pat. No. 4,521,548 describes how the polymerization-active centres can be deactivated in a similar manner by reaction with formic acid. The metal carboxylates formed after neutralization with hydroxycarboxylic acids or formic acid are soluble in the polyether polyols to give clear solutions. A disadvantage of these processes, however, is the catalytic activity, which is undesirable for many polyurethane uses, of the relatively basic carboxylic acid salts remaining in the products. In JP-A 10-30023 and U.S. Pat. No. 4,110,268 aromatic sulfonic acids or organic sulfonic acids are therefore employed for the neutralization, these likewise forming salts which are soluble in the polyether polyols but being less basic and distinguished by a lower catalytic activity. The high costs associated with the use of sulfonic acids are a decisive disadvantage here. Working up by means of acid cation exchangers, such as is described in DE-A 100 24 313, requires the use of solvents and separating off of these by distillation, and is therefore likewise associated with high costs. Phase separation processes require merely a hydrolysis step but no neutralization step and are described, for example, in WO 01/14456, JP-A 6-157743, WO 96/20972 and U.S. Pat. No. 3,823,145. The phase separation of the polyether polyols from the alkaline aqueous phase is assisted by the use of coalescers or centrifuges, and solvents must also often be added here in order to increase the density difference between the polyether phase and the aqueous phase. Such processes are not suitable for all polyether polyols, and they fail in particular in the case of short-chain polyether polyols or in the case of polyols with high ethylene oxide contents. The working up of polyether ester polyols likewise cannot be operated by such phase separation processes, since the long contact of the polyether ester chains with the alkaline aqueous phase would undeniably lead to irreversible hydrolysis of the ester bonds. The use of solvents is cost-intensive and centrifuges require a high outlay on maintenance.

A further working up can be dispensed with in the case of amine-catalysed alkylene oxide addition reactions if the presence of the amines in these polyols does not impair the preparation of polyurethane materials. Only polyols having relatively low equivalent weights can be obtained via amine catalysis, in this context see, for example, Ionescu et al. in “Advances in Urethane Science & Technology”, 1998, 14, p. 151-218.

The object of the present invention was therefore to find an inexpensive working up process for polyols which contain ethylene oxide and are prepared under alkali metal or alkaline earth metal hydroxide, carboxylate or hydride catalysis which does not have the disadvantages mentioned for the processes of the prior art.

It has been possible to achieve the object by carrying out the neutralization of the alkaline polymerization-active centres of the crude alkylene oxide addition product by addition of one or more monobasic inorganic acids. The alkylene oxide mixtures metered into the polyol preparation contains at least 10 wt. % of ethylene oxide, preferably at least 20 wt. % of ethylene oxide and particularly preferably more than 50 wt. % of ethylene oxide. Surprisingly, completely clear products with a low base content are obtained by this procedure without further working up steps. If instead of the monobasic inorganic acids the amounts of phosphoric acid (1 mol/mol of base) or sulfuric acid (0.5 mol/mol of base) sufficient for neutralization of the catalyst are employed, cloudy products with a high alkali content result. The method is applicable to long- and short-chain polyether polyols, i.e. the OH number range of the end products extends from about 20 mg of KOH/g to about 900 mg of KOH/g. The structure of the polyether chains, i.e. the composition of the alkylene oxide mixture employed, can likewise be varied in the polyol preparation process.

In detail, the process according to the invention is carried out as follows:

The starter compounds having zerewitinoff-active hydrogen atoms are conventionally initially introduced into the reactor and the catalyst, that is to say the alkali metal hydroxide, alkali metal or alkaline earth metal hydride, alkali metal or alkaline earth metal carboxylate or alkaline earth metal hydroxide, is added. Alkali metal hydroxides are preferably employed, and potassium hydroxide is particularly preferred. The catalyst can be added to the starter compound as an aqueous solution or as a solid. The catalyst concentration, based on the amount of end product, is 0.004 to 0.2 wt. %, preferably 0.004 to 0.07 wt. %. The solution water and/or the water liberated during the reaction of the zerewitinoff-active hydrogen atoms with the catalyst can be removed in vacuo at elevated temperature, preferably at the reaction temperature, before the start of metering of the alkylene oxide(s).

Basic catalysts which can also be employed are prefabricated alkylene oxide addition products of starter compounds containing zerewitinoff-active hydrogen atoms and having alkoxylate contents of from 0.05 to 50 equivalent % (“polymeric alkoxylates”). The alkoxylate content of the catalyst is to be understood as meaning the content of zerewitinoff-active hydrogen atoms removed by a base by deprotonation in all the zerewitinoff-active hydrogen atoms which were originally present in the alkylene oxide addition product of the catalyst. The amount of polymeric alkoxylates employed depends of course on the catalyst concentration sought for the amount of end product, as described in the preceding section.

The starter compounds initially introduced into the reactor are now reacted with alkylene oxides under an inert gas atmosphere at temperatures of 80-180° C., preferably at 100-170° C., the alkylene oxides being added to the reactor continuously in the usual manner such that the safety pressure limits of the reactor system used are not exceeded. Such reactions are conventionally carried out in the pressure range of from 10 mbar to 10 bar. The end of the alkylene oxide metering phase is usually followed by an after-reaction phase in which the remaining alkylene oxide reacts. The end of this after-reaction phase is reached when no further drop in pressure is detectable in the reaction tank. The alkaline alkylene oxide addition product can now be first hydrolysed by water. However, this hydrolysis step is not essential for carrying out the process according to the invention. The amount of water here is up to 15 wt. %, based on the amount of alkaline alkylene oxide addition products. Neutralization of the alkaline polymerization-active centres of the crude, optionally hydrolysed alkylene oxide addition product is then carried out by addition of one or more monobasic inorganic acids. The temperature during hydrolysis and neutralization can be varied within wide ranges, and limits can be given in this context by the corrosion resistance of the materials of the neutralization tank or the polyol composition. If groups which are sensitive to hydrolysis, such as, for example, ester groups, are present in the products, neutralization can be earned out, for example, at room temperature. In such cases it is advisable also to dispense with a prior separate hydrolysis step. When the neutralization has taken place, traces of water which have been introduced by addition of dilute acids and excess water of hydrolysis can be removed in vacuo. Anti-ageing agents or antioxidants can be added to the products during or after the neutralization. Further working up steps, such as, for example, filtration of the product, are not necessary.

Suitable starter compounds having zerewitinoff-active hydrogen atoms usually have functionalities of from 1 to 8, but also in certain cases functionalities of up to 35. Their molar masses are from 60 g/mol to 1,200 g/mol. In addition to hydroxy-functional starters, amino-functional starters can also be employed. Examples of hydroxy-functional starter compounds are methanol, ethanol, 1-propanol, 2-propanol and higher aliphatic mono-ols, in particular fatty alcohols, phenol, alkyl-substituted phenols, propylene glycol, ethylene glycol, diethylene glycol, dipropylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane, pentaerythritol, sorbitol, sucrose, hydroquinone, pyrocatechol, resorcinol, bisphenol F, bisphenol A, 1,3,5-trihydroxybenzene, condensates of formaldehyde and phenol or melamine or urea containing methylol groups, and Mannich bases. Highly functional starter compounds based on hydrogenated starch hydrolysis products can also be employed. Such compounds are described, for example, in EP-A 1 525 244. Examples of starter compounds containing amino groups are ammonia, ethanolamine, diethanolamine, triethanolamine, is opropanolamine, diisopropanolamine, ethylenediamine, hexamethylenediamine, aniline, the isomers of toluidine, the isomers of diaminotoluene, the isomers of diaminodiphenylmethane and products with a higher number of nuclei obtained in the condensation of aniline with formaldehyde to give diaminodiphenylmethane. Ring-opening products from cyclic carboxylic acid anhydrides and polyols can moreover also be employed as starter compounds. Examples are ring opening products from phthalic anhydride, succinic anhydride or maleic anhydride on the one hand and ethylene glycol, diethylene glycol, 1,2-butanediol, 1,3-butanediol, 1,4-butanediol, hexanediol, pentanediol, 3-methyl-1,5-pentanediol, 1,12-dodecanediol, glycerol, trimethylolpropane, pentaerythritol or sorbitol on the other hand. Mixtures of various starter compounds can of course also be employed.

Prefabricated alkylene oxide addition products of the starter compounds mentioned, that is to say polyether polyols having OH numbers of from 20 to 1,000 mg of KOH/g, preferably 250 to 1,000 mg of KOH/g, can furthermore also be added to the process. The alkylene oxide mixtures employed in the preparation of the prefabricated alkylene oxide addition products contain at least 10 wt. % of ethylene oxide, preferably at least 20 wt. % of ethylene oxide and particularly preferably more than 50 wt. % of ethylene oxide. It is also possible also to employ polyester polyols having OH numbers in the range of from 6 to 800 mg of KOH/g in the process according to the invention in addition to the starter compounds, with the aim of polyether ester preparation. Polyester polyols which are suitable for this can be prepared, for example, by known processes from organic dicarboxylic acids having 2 to 12 carbon atoms and polyhydric alcohols, preferably diols, having 2 to 12 carbon atoms, preferably 2 to 6 carbon atoms.

Against the background of the scarcity of petrochemical resources and the adverse rating of fossil raw materials in ecobalances, the use of raw materials from regenerable sources is also gaining increasing importance in the preparation of polyols which are suitable for the polyurethane industry. The process according to the invention opens up a highly economical possibility for the preparation of such polyols in that triglycerides, such as, for example, soya oil, rapeseed oil, palm kernel oil, palm oil, linseed oil, sunflower oil, herring oil sardine oil, lesquerella oil and castor oil, can be added to the process in amounts of 10-80 wt. %, based on the amount of end product, before or during the addition of the alkylene oxides. Polyether ester polyols into the structure of which the oils are incorporated completely, so that they can no longer be detected or can be detected in only very small amounts in the end product, are obtained.

The polymeric alkoxylates mentioned which can be used as the catalyst are prepared in a separate reaction step by addition of alkylene oxide on to starter compounds containing zerewitinoff-active hydrogen atoms. Conventionally, in the preparation of the polymeric alkoxylates, an alkali metal or alkaline earth metal hydroxide, e.g. KOH, is employed as a catalyst in amounts of from 0.1 to 1 wt. %, based on the amount to be prepared, the reaction mixture, if necessary, is dewatered in vacuo, the alkylene oxide addition reaction is carried out under an inert gas atmosphere at 100 to 170° C. until an OH number of from 150 to 1,200 mg of KOH/g is reach, and thereafter, if appropriate, the product is adjusted to the abovementioned alkoxylate contents of from 0.05 to 50 equivalent % by addition of further alkali metal or alkaline earth metal hydroxide and subsequent dewatering. Polymeric alkoxylates prepared in such a manner can be stored separately under an inert gas atmosphere. They are particularly preferably used in the process according to the invention if starting substances which are sensitive to hydrolysis under alkaline conditions are used or the amount of low molecular weight starter in the preparation of long-chain polyols is not sufficient to ensure adequate thorough mixing of the reaction mixture at the start of the reaction. Furthermore, certain low molecular weight starters tend to form sparingly soluble alkali metal or alkaline earth metal salts; in such cases prior conversion of the starter into a polymeric alkoxylate by the process described above is likewise advisable. The amount of polymeric alkoxylate employed in the process according to the invention is conventionally such that it corresponds to an alkali metal or alkaline earth metal hydroxide concentration, based on the amount of end product according to the invention to be prepared, of from 0.004 to 0.2 wt. %, preferably 0.004 to 0.07 wt. %. The polymeric alkoxylates can of course also be employed as mixtures.

Suitable alkylene oxides are, for example, ethylene oxide, propylene oxide, 1,2-butylene oxide or 2,3-butylene oxide and styrene oxide. Propylene oxide and ethylene oxide are preferably employed. As already mentioned, preferably at least 10, particularly preferably at least 20 and very particularly preferably more than 50 wt. % of ethylene oxide, based on the total amount of metered alkylene oxides, should be present. If the polymeric alkoxylates mentioned in the preceding section are used as catalysts, or the abovementioned prefabricated alkylene oxide addition products are employed as components in the starter mixture, the amounts of ethylene oxide used in their preparation are also included in the calculation of the total ethylene oxide content. The ethylene oxide can be metered in a mixture with the other alkylene oxides or blockwise as the initial, middle or end block. Products with ethylene oxide end blocks are characterized, for example, by increased concentrations of primary end groups, which impart to the systems the isocyanate reactivity necessary for moulded foam uses.

The alkaline crude polyols in general have OH numbers of from 20 to 1,000 mg of KOH/g, preferably OH numbers of from 28 to 700 mg of KOH/g.

The working up of the alkaline crude polyols is carried out by addition of monobasic inorganic acids, such as, for example, hydrogen halide acids, nitric acid, iodic acid, periodic acid, bromic acid, chloric acid or perchloric acid. Perchloric acid, the hydrogen halide acids and nitric acid are preferred. The neutralization can be carried out at the reaction temperature. Stoichiometric amounts of the monobasic inorganic acids with respect to the amount of catalyst to be neutralized are preferably employed. If functional groups which are sensitive to hydrolysis, such as, for example, ester groups, are present, the neutralization can also be carried out at a significantly lower temperature, for example at room temperature. The water introduced with the neutralizing acid(s) can be removed in vacuo. Further working up steps, such as, for example, filtration of the product, are not necessary.

The actual neutralization can be preceded by a separate hydrolysis step, but this is not of essential importance for the process according to the invention. The amount of water in such a hydrolysis step is up to 15 wt. %, based on the amount of the alkaline alkylene oxide addition product.

The polyols obtainable by the process according to the invention can be employed as starting components for the preparation of solid or foamed polyurethane materials and of polyurethane elastomers. The polyurethane materials and elastomers can also contain isocyanurate, allophanate and biuret structural units. The preparation of so-called isocyanate prepolymers for the preparation of which a molar ratio of isocyanate groups to hydroxyl groups of greater than 1 is used, so that the product contains free isocyanate functionalities, is likewise possible. The latter are first reacted in one or more steps during the preparation of the actual end product.

For the preparation of these materials, the polyols according to the invention are optionally mixed with further isocyanate-reactive components and reacted with organic polyisocyanates, optionally in the presence of blowing agents in the presents of catalysts, optionally in the presence of other additives, such as e.g. cell stabilizers.

### EXAMPLES

### Raw Materials Employed:

Soya oil: Soya oil (refined, i.e. delecithinated, neutralized, decolorized and vapour-stripped), obtained from Sigma-Aldrich Chemie GmbH, Munich.

### Preparation of Polymeric Alkoxylate 1:

1,278.5 g of trimethylolpropane and 21.7 g of a 45 wt. % strength solution of KOH in water were introduced into a 10 l laboratory autoclave under a nitrogen atmosphere. The autoclave was closed, the stirrer speed was adjusted to 450 rpm and the mixture was heated up to 107° C. The pressure was lowered to 100 mbar and 653.4 g of propylene oxide were metered into the autoclave over a period of 3 h. After an after-reaction time of 30 min at 107° C., the mixture was heated thoroughly in vacuo for 30 min. After cooling to room temperature, 45.1 g of a 45 wt. % strength solution of KOH in water were added under a nitrogen atmosphere. The mixture was heated to 107° C. and the water was removed in vacuo, until a pressure of 10 mbar was reached. 4,063.6 g of propylene oxide were then metered in at 107° C. over a period of 8.5 h, and after an after-reaction time of 120 min the mixture was heated thoroughly in vacuo for 30 min. After cooling to room temperature, 539.4 g of a 45 wt. % strength solution of KOH in water were added under a nitrogen atmosphere. The mixture was heated to 107° C. and the water was removed in vacuo until a pressure of 10 mbar was reached. The alkali number of polymeric alkoxylate 1 is 44.1 mg of KOH/g, its OH number is 260 mg of KOH/g. The alkoxylate content is 17%.

Preparation of Polymeric Alkoxylate 2:

3,677.2 g of glycerol and 18.65 g of a 45 wt. % strength solution of KOH in water were introduced into a 10 l laboratory autoclave under a nitrogen atmosphere. The autoclave was closed, the stirrer speed was adjusted to 450 rpm and the mixture was heated up to 110° C. The pressure was lowered to 100 mbar and 2,313.7 g of propylene oxide were metered into the autoclave over a period of 4.6 h. After an after-reaction time of 180 min at 110° C., the pressure was lowered slowly again to 100 mbar and the mixture was finally freed from water in vacuum until a pressure of less than 10 mbar was reached at a temperature of 110° C. The alkali number of polymeric alkoxylate 2 is 1.4 mg of KOH/g, its OH number is 1,121 mg of KOH/g. The alkoxylate content is 0.125%.

Irganox® 1076: Octadecyl 3-(3,5-di-tert-butyl-1,4-hydroxyphenyppropionate

Neutralization Method:

In all the examples and comparison examples, exactly the amount of acid needed for 100% neutralization of the base (KOH) was employed, that is to say 1 mol of acid/mol of KOH in the case of the monobasic inorganic acids to be used according to the invention and phosphoric acid or 0.5 mol of sulfuric acid/mol of KOH.

### Example 1 (Comparison)

27.6 g of polymeric alkoxylate 1 and 1,213.0 g of trimethylolpropane were introduced into a 10 l laboratory autoclave under a nitrogen atmosphere. The autoclave was closed and charged with a nitrogen pressure of 1.5 bar. The mixture was heated to 150° C., while stirring (450 rpm), and 4,646.9 g of ethylene oxide were metered into the autoclave at a stirrer speed of likewise 450 rpm over a period of 13.2 h. During this metering time, a reactor pressure of 4 bar was meanwhile reached, the mixture was then allowed to react for 25 min, the pressure was lowered by letting off the nitrogen to 2.55 bar and the ethylene oxide metering was resumed. After the end of the ethylene oxide metering, an after-reaction time of 45 min duration followed. The mixture was heated thoroughly in vacuo for 30 min and thereafter cooled to room temperature. The catalyst concentration calculated for KOH was 200 ppm.

75 g of distilled water, 1.4571 g of 11.83% strength sulfuric acid and 1.038 g of Irganox® 1076 were added to 936 g of the product and the mixture was stirred at 80° C. for 1 h. Thereafter, the produce was dewatered under 18 mbar (water jet vacuum) for 1 h and then under 1 mbar at 110° C. for 3 h. Thereafter, the product showed slight clouding.

### Example 2 (Comparison)

5.49 g of polymeric alkoxylate 1 and 243.7 g of trimethylolpropane were introduced into a 2 l laboratory autoclave under a nitrogen atmosphere. The autoclave was closed and its contents were heated to 150° C., while stirring (700 rpm). 956.9 g of ethylene oxide were metered into the autoclave at a stirrer speed of likewise 700 rpm over a period of 6 h. Towards the end of the ethylene oxide metering phase, a reactor pressure of 1.9 bar was reached. After the end of the ethylene oxide metering an after-reaction time of 20 minutes followed. The mixture was heated thoroughly in vacuo for 30 min. The catalyst concentration calculated for KOH was 200 ppm.

BMS 08 1 024

56 g of distilled water and 5.9675 g of 3.293% strength phosphoric acid were added to 556.5 g of the produce at 80° C. and the mixture was stirred at 80° C. for ½ h. After addition of 0.622 g of Irganox® 1076, the product was dewatered under 18 mbar (water jet vacuum) for 1 h and then under 1 mbar at 110° C. for 3 h. A cloudy product was obtained.

### Example 3

13.56 g of polymeric alkoxylate 1 and 1,215.2 g of trimethylolpropane were introduced into a 10 l laboratory autoclave under a nitrogen atmosphere. The autoclave was closed and charged with a nitrogen pressure of 1.9 bar. The mixture was heated to 150° C., while stirring (450 rpm), and 4,771.5 g of ethylene oxide were metered into the autoclave at a stirrer speed of likewise 450 rpm over a period of 10.8 h. During this metering time, metering was interrupted 3 times, the mixture was allowed to react, the pressure was lowered by letting off the nitrogen to 2.5 bar and the ethylene oxide metering was then resumed. After the end of the ethylene oxide metering, an after-reaction time of 1 h duration followed. After a thorough heating time of 30 min in vacuo and cooling to room temperature, 2 portions of the mixture were taken for neutralization experiments (Examples 3A and 3B). The catalyst concentration calculated for KOH was 100 ppm.

### Example 3A (Comparison)

0.206 g of 85% strength phosphoric acid were added to 987.1 g of the product from Example 3 at 80° C. under a nitrogen atmosphere and the mixture was stirred at 80° C. for 1 h. After addition of 0.495 g of Irganox® 1076, the product was dewatered under 18 mbar (water jet vacuum) for 1 h and then under 1 mbar at 110° C. for 3 h. A slightly cloudy product was obtained.

### Example 3B

1.818 g of hydrochloric acid (concentration: 1 mol/kg) were added to 1,023.1 g of the product from Example 3 at 80° C. under a nitrogen atmosphere and the mixture was stirred at 80° C. for 1 h. After addition of 0.516 g of Irganox® 1076, the product was dewatered under 18 mbar (water jet vacuum) for 1 h and then under 1 mbar at 110° C. for 3 h. A clear product was obtained.

### Example 4

64.2 g of polymeric alkoxylate 1 and 1,202.9 g of trimethylolpropane were introduced into a 10 l laboratory autoclave under a nitrogen atmosphere. The autoclave was closed and charged with a nitrogen pressure of 1.5 bar. The mixture was heated to 150° C., while stirring (450 rpm), and 4,733.3 g of ethylene oxide were metered into the autoclave at a stirrer speed of likewise 450 rpm over a period of 14.23 h. During this metering time, a reactor pressure of 4 bar was meanwhile reached; the mixture was then allowed to react for 25 min, the pressure was lowered by letting off the nitrogen to 2.55 bar and the ethylene oxide metering was resumed. After the end of the ethylene oxide metering, an after-reaction time of 31 min duration followed. After a thorough heating phase of 30 min duration in vacuo and cooling to room temperature, the mixture was divided into three portions (Examples 4A, 4B and 4C) for neutralization experiments. The catalyst concentration calculated for KOH was 470 ppm.

### Example 4A (Comparison)

169 ml of distilled water and 5.813 g of 11.89% strength sulfuric acid were added to 1,683.8 g of the product from Example 4 at 80° C. under a nitrogen atmosphere and the mixture was stirred at 80° C. for 1 h. After addition of 1.857 g of Irganox® 1076, the product was dewatered under 18 mbar (water jet vacuum) for 1 h and then under 1 mbar at 110° C. for 3 h. A cloudy product was obtained.

### Example 4B

8.951 g of 20% strength perchloric acid were added to 2,127.8 g of the product from Example 4 at 80° C. under a nitrogen atmosphere and the mixture was stirred at 80° C. for 1 h. After addition of 2.340 g of Irganox® 1076, the product was dewatered under 18 mbar (water jet vacuum) for 1 h and then under 1 mbar at 110° C. for 3 h. a clear product was obtained.

### Example 4C

17.91 g of hydrochloric acid (concentration: 1 mol/kg) were added to 2,130.4 g of the product from Example 4 at 80° C. under a nitrogen atmosphere and the mixture was stirred at 80° C. for 1 h. After addition of 2.349 g of Irganox® 1076, the product was dewatered under 18 mbar (water jet vacuum) for 1 h and then under 1 mbar at 110° C. for 3 h. A clear product was obtained.

### Example 5

1,200 g of polymeric alkoxylate 2 and 79.8 g of glycerol were introduced into a 10 l laboratory autoclave under a nitrogen atmosphere. The autoclave was closed, the mixture was heated to 150° C., while stirring (450 rpm), and the autoclave was charged with a nitrogen pressure of 2.7 bar. 4,721.6 g of ethylene oxide were metered into the autoclave at a stirrer speed of likewise 450 rpm over a period of 8.1 h. During this metering time, the metering was interrupted once, the mixture was allowed to react, the pressure was lowered by letting off the nitrogen to 2.5 bar and the ethylene oxide metering was then resumed. After the end of the ethylene oxide metering, an after-reaction time of 1 h duration followed. After a thorough heating phase of 30 min in vacuo and cooling to room temperature, the mixture was divided into three portions (Examples 5A, 5B and 5C) for neutralization experiments. The catalyst concentration calculated for KOH was 280 ppm.

### Example 5A (Comparison)

3.169 g of 11.89% strength sulfuric acid were added to 1,523.2 g of the product from

Example 5 at 80° C. under a nitrogen atmosphere and the mixture was stirred at 80° C. for 1 h. After addition of 0.750 g of Irganox® 1076, the product was dewatered under 18 mbar (water jet vacuum) for 1 h and then under 1 mbar at 110° C. for 3 h. A slightly cloudy product was obtained.

### Example 5B

7.407 g of hydrochloric acid (concentration: 1 mol/kg) were added to 1,461.8 g of the product from Example 5 at 80° C. under a nitrogen atmosphere and the mixture was stiffed at 80° C. for 1 h. After addition of 0.744 g of Irganox® 1076, the product was dewatered under 18 mbar (water jet vacuum) for 1 h and then under 1 mbar at 110° C. for 3 h. A clear product was obtained.

### Example 5C

3.463 g of 20.35% strength perchloric acid were added to 1,372.3 g of the product from Example 5 at 80° C. under a nitrogen atmosphere and the mixture was stirred at 80° C. for 1 h. After addition of 0.705 g of Irganox® 1076, the product was dewatered under 18 mbar (water jet vacuum) for 1 h and then under 1 mbar at 110° C. for 3 h. A clear product was obtained.

### Example 6

325.5 g of sorbitol and 3.209 g of a 44.82% strength solution of KOH in water were introduced into a 10 l laboratory autoclave under a nitrogen atmosphere. The autoclave was closed and its contents were stripped with 50 ml of nitrogen per minute in vacuo at 110° C. over a period of 3 h and at a stirrer speed of 450 rpm. The mixture was heated to 150° C., while stirring (450 rpm), and 1,135.1 g of propylene oxide were metered into the autoclave over a period of 3.22 h such that a constant pressure of 5 bar was reached. After an after-reaction time of 2.45 h, the reactor pressure was adjusted to 2.7 bar with nitrogen and 4,540.2 g of ethylene oxide were metered in over a period of 9.07 h. During this metering time, the metering was interrupted twice when a reactor pressure of 5 bar was reached, the mixture was allowed to react each time, the pressure was lowered by letting off the nitrogen to 2.5 bar and the alkylene oxide metering was then resumed. After the end of the ethylene oxide metering, an after-reaction time of 1.5 h duration followed. After a thorough heating time of 30 min in vacuo, the mixture was cooled to room temperature. The catalyst concentration calculated for KOH was 240 ppm. 5.300 g of 10.35% strength nitric acid were added to 2,033.1 g of the product at 80° C. and the mixture was stirred at 80° C. for 0.1 h. After addition of 1.018 g of Irganox® 1076, the product was dewatered under 1 mbar at 110° C. for 3 h. A clear product was obtained.

### Example 7

821.9 g of glycerol and 1.609 g of a 44.82% strength solution of KOH in water were introduced into a 10 l laboratory autoclave under a nitrogen atmosphere. The autoclave was closed and its contents were stripped with 50 ml of nitrogen per minute in vacuo at 110° C. over a period of 3 h and at a stirrer speed of 450 rpm. The mixture was heated to 150° C., while stirring (450 rpm), and 4,150.1 g of propylene oxide were metered into the autoclave over a period of 11.7 h such that a constant pressure of 5 bar was reached. After the end of the propylene oxide metering phase, an after-reaction time of 3 h duration followed. The reactor was then charged with a nitrogen pressure of 2.5 bar and 1,037.5 g of ethylene oxide were metered in over a period of 4.25 h. After the end of the ethylene oxide metering, an after-reaction time of 1.5 h duration followed. After a thorough heating time of 30 min in vacuo and cooling to room temperature, three portions were taken from the mixture for neutralization experiments (Examples 7A, 7B and 7C). The catalyst concentration calculated for KOH was 120 ppm.

### Example 7A

1.449 g of 10.35% strength nitric acid were added to 1,117.3 g of the product from Example 7 at 80° C. under a nitrogen atmosphere and while stirring and the mixture was stirred at 80° C. for 1 h. After addition of 0.563 g of Irganox® 1076, the product was dewatered under 18 mbar (water jet vacuum) for 1 h and then under 1 mbar at 110° C. for 3 h. A clear product was obtained.

### Example 7B

136.3 g of water and thereafter 1.4351 g of 20.35% strength perchloric acid were added to 1,362.9 g of the product from Example 7 at 80° C. under a nitrogen atmosphere and while stirring and the mixture was stirred at 80° C. for 1 h. After addition of 0.688 g of Irganox® 1076, the product was dewatered under 18 mbar (water jet vacuum) for 1 h and then under 1 mbar at 110° C. for 3 h. A clear product was obtained.

### Example 7C (Comparison)

1,002 g of 11.80% strength sulfuric acid were added to 1,129.3 g of the product from Example 7 at 80° C. under a nitrogen atmosphere and while stirring and the mixture was stiffed at 80° C. for 1 h. After addition of 0.567 g of Irganox® 1076, the product was dewatered under 18 mbar (water jet vacuum) for 1 h and then under 1 mbar at 110° C. for 3 h. A slightly cloudy product was obtained.

### Example 8

820.7 g of glycerol and 1.472 g of a 44.82 wt. % strength solution of KOH in water were introduced into a 10 l laboratory autoclave under a nitrogen atmosphere. The autoclave was closed and its contents were stripped with 50 ml of nitrogen per minute in vacuo at 110° C. over a period of 3 h and at a stirrer speed of 450 rpm. The mixture was heated to 150° C., while stirring (450 rpm), and the autoclave was charged with a nitrogen pressure of 1.7 bar. A mixture of 3,884.5 g of ethylene oxide and 1,289.2 g of propylene oxide was metered into the autoclave at 150° C. at a stirrer speed of likewise 450 rpm over a period of 10.53 h. During this metering time, the metering was interrupted twice when a reactor pressure of 5 bar was reached, the mixture was allowed to react each time, the pressure was lowered by letting off the nitrogen to 1.7 bar and the alkylene oxide metering was then resumed. After the alkylene oxide metering phase, an after-reaction time of 3.25 h duration followed. After a thorough heating time of 30 min in vacuo and cooling to room temperature, two portions were taken from the mixture for neutralization experiments (Examples 8A and 8B). The catalyst concentration calculated for KOH was 110 ppm.

### Example 8A

2.589 g of 10.35% strength nitric acid were added to 2,148.2 g of the product from Example 8 at 80° C. under a nitrogen atmosphere and while stirring and the mixture was stirred at 80° C. for 1 h. After addition of 1.081 g of Irganox® 1076, the product was dewatered under 18 mbar (water jet vacuum) for 1 h and then under 1 mbar at 110° C. for 3 h. A clear product was obtained.

### Example 8B

1.3712 g of 20.35% strength perchloric acid were added to 1,295.1 g of the product from Example 8 at 80° C. under a nitrogen atmosphere and while stirring and the mixture was stirred at 80° C. for 1 h. After addition of 0.658 g of Irganox® 1076, the product was dewatered under 18 mbar (water jet vacuum) for 1 h and then under 1 mbar at 110° C. for 3 h. A clear product was obtained.

### Example 9

1,817.6 g of trimethylolpropane and 4.042 g of a 44.8 wt. % strength solution of KOH in water were introduced into a 10 l laboratory autoclave under a nitrogen atmosphere. The autoclave was closed and its contents were stripped with 50 ml of nitrogen per minute in vacuo at 110° C. over a period of 3 h and at a stirrer speed of 450 rpm. The mixture was then heated to 150 ° C. and 4,182.4 g of propylene oxide were metered into the autoclave at a stirrer speed of likewise 450 rpm over a period of 10.6 h. The propylene oxide metering was started under a pressure of 1.1 bar, and during the metering phase a pressure of 4.95 bar was reached. After the end of the propylene oxide metering, an after-reaction time of 7 h duration followed. After a thorough heating time of 30 min in vacuo and cooling to room temperature, two portions were taken from the mixture for neutralization experiments (Examples 9A and 9B). The catalyst concentration (KOH) was 300 ppm.

### Example 9A (Comparison)

7.930 g of 10.35% strength nitric acid were added to 2,274 g of the product from Example 9 at 80° C. under a nitrogen atmosphere and the mixture was stirred at 80° C. for 1 h. After addition of 1.140 g of Irganox® 1076, the product was dewatered under 18 mbar (water jet vacuum) for 1 h and then under 1 mbar at 110° C. for 3 h. A cloudy product was obtained.

### Example 9B (Comparison)

6.126 g of 20.35% strength perchloric acid were added to 2,332 g of the product from Example 9 at 80° C. under a nitrogen atmosphere and the mixture was stirred at 80° C. for 1 h. After addition of 1.160 g of Irganox 1076, the product was dewatered under 18 mbar (water jet vacuum) for 1 h and then under 1 mbar at 110° C. for 3 h. A cloudy product was obtained.

### Example 10

688.5 g of sorbitol and 2.547 g of a 44.82 wt. % strength solution of KOH in water were introduced into a 10 l laboratory autoclave under a nitrogen atmosphere. The autoclave was closed and its contents were stripped with 50 ml of nitrogen per minute in vacuo at 110° C. over a period of 3 h and at a stirrer speed of 450 rpm. 3,298.3 g of soya oil were added at about 110° C. under a nitrogen atmosphere and the mixture was heated to 130° C., while stirring (450 rpm). The pressure was adjusted to 2.5 bar with nitrogen. 2,021.5 g of ethylene oxide were metered into the autoclave at 130° C. over a period of 7.15 h such that a total pressure of 5 bar was not exceeded. After the end of the ethylene oxide metering, an after-reaction time of 5.95 h duration followed. The catalyst concentration (KOH) was 190 ppm. After a thorough heating phase of 30 min in vacuo and cooling to room temperature, two portions were taken from the mixture for neutralization experiments (Examples 10A and 10B).

### Example 10A

7.425 g of hydrochloric acid (concentration: 1 mol/kg) were added to 2,162 g of the product from Example 10 at 40° C. under a nitrogen atmosphere and the mixture was stirred at 40° C. for 1 h. After addition of 1.084 g of Irganox® 1076, the product was dewatered under 1 mbar at 110° C. for 3 h. A clear product was obtained.

### Example 10B

3.154 g of 20.35% strength perchloric acid were added to 1,889 g of the product from Example 10 at 40° C. under a nitrogen atmosphere and the mixture was stirred at 40° C. for 1 h. After addition of 0.945 g of Irganox® 1076, the product was dewatered under 1 mbar at 110° C. for 3 h. A clear product was obtained.

### Example 11

820.9 g of glycerol and 1.742 g of a 44.82 wt. % strength solution of KOH in water were introduced into a 10 l laboratory autoclave under a nitrogen atmosphere. The autoclave was closed and its contents were stripped with 50 ml of nitrogen per minute in vacuo at 150° C. over a period of 3 h and at a stirrer speed of 450 rpm. 1,035.9 g of ethylene oxide were metered into the autoclave at a stirrer speed of likewise 450 rpm over a period of 2.67 h. The ethylene oxide addition was started under a nitrogen pressure of 2.55 bar, and during the metering phase a maximum pressure of 4 bar was reached. After an after-reaction time of 1.52 h, the pressure was lowered to 0.05 bar. 4,143.5 g of propylene oxide were metered in at likewise 150° C. in the course of 10.25 h, and the maximum reactor pressure reached during the procedure was 5 bar. After the end of the propylene oxide metering, an after-reaction time of 11 h duration followed. After a thorough heating time of 30 min in vacuo and cooling to room temperature, three portions were taken from the mixture for neutralization experiments (Examples 11A, 11B and 11C). The catalyst concentration (KOH) was 130 ppm.

### Example 11A

1.749 g of 20.35 wt. % strength perchloric acid were added to 1,516.3 g of the product from Example 11 at 80° C. under a nitrogen atmosphere and the mixture was stirred at 80° C. for 1 h. After addition of 0.76 g of Irganox® 1076, the product was dewatered under 18 mbar (water jet vacuum) for 1 h and then under 1 mbar at 110° C. for 3 h. A clear product was obtained.

### Example 11B

1.918 g of 10.35 wt. % strength nitric acid were added to 1,352.7 g of the product from Example 11 at 80° C. under a nitrogen atmosphere and the mixture was stirred at 80° C. for 1 h. After addition of 0.682 g of Irganox® 1076, the product was dewatered under 18 mbar (water jet vacuum) for 1 h and then under 1 mbar at 110° C. for 3 h. A clear product was obtained.

### Example 11C

3.03 g of hydrochloric acid (concentration: 1 mol/kg) were added to 1,277.6 g of the product from Example 11 at 80° C. under a nitrogen atmosphere and the mixture was stirred at 80° C. for 1 h. After addition of 0.646 g of Irganox® 1076, the product was dewatered under 18 mbar (water jet vacuum) for 1 h and then under 1 mbar at 110° C. for 3 h. A clear product was obtained.

Tab. 1 summarizes the analytical data of the polyols prepared in the examples.

TABLE 1

| | Base content | Acid number | OH number | |
|---|---|---|---|---|
| | measured | measured | measured | |
| | [ppm of | [ppm of | [mg of | Appear- |
| Example | KOH] | KOH] | KOH/g] | ance |
| 1 (comparison) | 97.8 | 99 | 256 | cloudy |
| 2 (comparison) | 187.2 | 153 | 258 | cloudy |
| 3A (comparison) | 95.1 | 11.3 | 257 | slightly |
| | | | | cloudy |
| 3B | 1.0 | 22.7 | 257 | clear |
| 4A (comparison) | 81.2 | 46.1 | 254 | cloudy |
| 4B | 14.1 | 42.2 | 255 | clear |
| 4C | 16.2 | 41.8 | 255 | clear |
| 5A (comparison) | 128.3 | 16.4 | 253 | cloudy |
| 5B | 1.0 | 16.0 | 253 | clear |
| 5C | 0.0 | 24.9 | 253 | clear |
| 6 | 5.0 | 27.0 | 100.4 | clear |
| 6B | 2.0 | 17.0 | 253 | clear |
| 7A | 10.0 | 8.0 | 248 | clear |
| 7B | 3.5 | 16.0 | 249 | clear |
| 7C (comparison) | 61.0 | 10.0 | 248 | cloudy |
| 8A | 2.0 | 5.0 | 248 | clear |
| 8B | 0.2 | 6.0 | 248 | clear |
| 9A (comparison) | 1.0 | 132.0 | 379 | cloudy |
| 9B (comparison) | 8.0 | 135.0 | 379 | cloudy |
| 10A | 20.0 | 127.0 | 209 | clear |
| 10B | 0.2 | 164.0 | 208 | clear |
| 11A | 0.1 | 12.0 | 251 | clear |
| 11B | 5.0 | 0.1 | 251 | clear |
| 11C | 5.0 | 1.0 | 251 | clear |

1-5. (canceled)

6. A process for preparing a polyol comprising reacting alkylene oxides via base-catalysed addition with polyfunctional starter compounds having zerewitinoff-active hydrogen atoms in the presence of catalysts based on alkali metal hydroxides, alkali metal or alkaline earth metal hydrides, alkali metal or alkaline earth metal carboxylates, or alkaline earth metal hydroxides in a concentration of from 0.004 to 0.2 weight %, based on the amount of end product, wherein the content of ethylene oxide in said alkylene oxides is at least 10 weight % and wherein neutralization of the alkaline polymerization-active centres of the crude alkylene oxide addition product is carried out by addition of monobasic inorganic acids.

7. The process of claim 6, wherein said process does not comprise separating off of any salts formed.

8. The process of claim 6, wherein from 10 to 80 weight %, based on the amount of end product, of triglycerides are added before or during addition of said alkylene oxides.

9. A polyol prepared according to the process of claim 6.

10. A polyurethane prepared from the polyol prepared according to the process of claim 6.
